(19) United States
(12) Patent Application Publication
(10) Pub. No.: US 2013/0109966 A1
(43) Pub. Date: May 2, 2013

(54) CONTROL UNIT AND MEDICAL EXAMINATION APPARATUS HAVING A CONTROL UNIT

(71) Applicants: Stefan Assmann, Erlangen (DE); Björn Heismann, Erlangen (DE); Reto Merges, Erlangen (DE); Markus Schmidt, Nuernberg (DE); Sebastian Schmidt, Weisendorf (DE); Kera Westphal, Berlin (DE)

(72) Inventors: Stefan Assmann, Erlangen (DE); Björn Heismann, Erlangen (DE); Reto Merges, Erlangen (DE); Markus Schmidt, Nuernberg (DE); Sebastian Schmidt, Weisendorf (DE); Kera Westphal, Berlin (DE)

(21) Appl. No.: 13/664,531

(22) Filed: Oct. 31, 2012

(30) Foreign Application Priority Data
Nov. 2, 2011 (DE) 102011085618.8

(51) Int. Cl. A61B 6/00

(52) U.S. Cl. 600/431

(57) ABSTRACT

Disclosed is a control unit for a device arrangement. The control unit includes an image-generating modality and a controllable injection apparatus for a contrast agent. The injection rate of the injection apparatus may be varied during data acquisition as a function of a patient-specific cycle duration.

### CROSS REFERENCE TO RELATED APPLICATIONS

This application claims priority of German Patent Office application No. 10 2011 085 618.8 DE filed Nov. 2, 1011. All of the applications are incorporated by reference herein in their entirety.

### FIELD OF INVENTION

A control unit and a medical examination apparatus are disclosed.

### BACKGROUND OF INVENTION

When patients are being examined using an image-generating modality, such as a computed tomography unit or a magnetic resonance tomography unit for example, contrast agents are frequently used, to manipulate the image signals generated by means of the image-generating modality. The corresponding contrast agent is injected for example into a vein for this purpose, typically a vein in the arm, whereupon the contrast agent mixes with the patient's blood. Such mixing initially is locally limited, so that a sort of concentrated package of contrast agent initially moves forward in the manner of a shockwave in the patient's bloodstream, gradually dispersing as it travels further through the bloodstream. Such a concentrated package of contrast agent is also referred to medically as a bolus.

The contrast agent is frequently injected at a relatively high and uniform flow rate, as a result of which the concentration of contrast agent in the corresponding blood vessel has a spatial distribution like a narrow bell curve, so that the contrast agent has the desired significant influence on image signal generation in an organ to be examined at least for a short time. One disadvantage here is the fact that the time window, in which image generation is favorably influenced by the contrast agent in the region to be examined is very narrow and the dispersal of the concentrated package of contrast agent as it passes through the blood steam means that the maximum concentration and/or the gradient of the spatial distribution of the concentration of contrast agent during the second pass of the organ being examined or the first bolus echo is no longer adequate to influence image signal generation expediently. Since an arbitrary quantity of the corresponding contrast agent cannot be administered, it is desirable for the intended generation of image data to take place during the first pass of the bolus through the region to be examined. However this is not always possible.

The desired information acquisition is also problematic because standard contrast agents penetrate the vessel walls to some degree and become lodged in the surrounding tissue. Even though the lodged contrast agent ultimately breaks down after a certain period in the tissue, it means that for the period when it is lodged, it is difficult to distinguish between tissue and vessels, as in an overexposed photograph. This is particularly unfavorable for examinations such as angiography or perfusion examinations for example, in which the movement of the bolus through the region to be examined, for example an organ, and therefore the change in the influence of the contrast agent on the image signals over time, is the object of the examination. To get around this problem, so-called “blood pool agents” are sometimes used. These are special contrast agents, which do not penetrate the vessel walls. However patients are less able to tolerate these special contrast agents and the special contrast agents are generally much more expensive than conventional contrast agents. The problem of the dispersal of the concentrated contrast agent package is also not resolved.

A method for producing tomographic images using contrast agent injections is known from DE 10 2005 041 626 A1, in which a patient is first injected with a defined test bolus, with the pattern of the concentration of the contrast agent over time being determined in at least one scan plane in at least one predefined body region, in order to determine function parameters of a prediction model from the measured pattern of contrast agent concentration in relation to the pattern of the test bolus injection. This prediction model is then used as a basis for subsequent examinations.

A method for controlling an image-generating process is also described in US 2002/0165445 A1, in which the injection of a contrast agent is controlled on the basis of data generated by an image-generating unit.

### SUMMARY OF INVENTION

A control unit and a medical examination apparatus, which can be used to deploy a contrast agent in an even more expedient manner are disclosed.

The control unit described below is intended for a device arrangement, which comprises an image-generating modality and a controllable injection apparatus for a contrast agent, the control unit being set up in such a manner that the injection rate of the injection apparatus may be varied and is varied during data acquisition as a function of a patient-specific cycle duration. The device arrangement here may be a medical examination apparatus and the image-generating modality is for example a computed tomography unit or a magnetic resonance tomography unit. Data acquisition then corresponds essentially to an examination of a patient with the aid of the image-generating modality, in other words for example the magnetic resonance tomography unit, and the patient-specific cycle duration refers in particular to the time taken by a relevant blood volume to be pumped once through the bloodstream of the patient. The control unit may be used automatically to manipulate the bolus shape, in other words the spatial distribution of the concentration of contrast agent, or, from another point of view, the distribution of the concentration of contrast agent in the blood of the patient over time, as part of the patient examination, so that image generation is influenced particularly favorably with the aid of the image-generating modality.

A multiple-stage injection of contract agent may be provided such as a two-stage injection of contrast agent. In principle each of the two stages corresponds to an injection of a partial dose of contrast agent and in which the time interval between the two stages is defined by the patient-specific cycle duration. When the first stage of the injection has taken place, the resulting bolus migrates through the bloodstream in the body of the patient, until it is finally back at the position where the injection took place, at which point the second injection stage is automatically started. The second injection stage therefore reshapes the bolus of the first injection stage, as it were reinforcing or refreshing it. This to some extent compensates for the dispersal of the bolus as it travels through the bloodstream of the patient, whereupon a region to be examined is subjected twice to an image-generating process as part of an examination, with the contrast agent influencing image generation in the desired manner in both instances. A physician is thus able to acquire much more data for a subsequent diagnosis during an examination. The control unit is therefore configured for automatic control of contrast agent administration.

The refreshing of the bolus means that much less contrast agent has to be injected to generate a specified bolus amplitude and/or to predefine a specified gradient for the distribution of the concentration of contrast agent than is the case when generating a new second bolus. The strain on the body of the patient is therefore reduced correspondingly by the reduced quantity of contrast agent, to which it is exposed. It is also ensured at the same time that the data acquired during the examination is suitable as a basis for a subsequent diagnosis, even if one of the two image-generation processes has been unfavorable during the course of said examination.

An embodiment of the control unit is also expedient, in which the patient-specific cycle duration is predefined for the respective data acquisition. According to one particularly simple embodiment of the control unit a value table is stored for example in a data storage unit, from which the control unit selects a value for the cycle duration based on patient-specific information, such as the patient's weight, age and sex, to use in the context of the corresponding data acquisition. The patient-specific cycle duration does not have to be determined using measuring technology in this very simple instance, with the result that the technical outlay for the device arrangement, in other words in particular the medical examination apparatus, is kept low.

As an alternative to using stored values for the cycle duration, provision is made for using a measuring apparatus to determine the patient-specific cycle duration for the respective data acquisition. The determination of the cycle duration here does not necessarily take place directly based on the measurement data determined using the measuring apparatus but serves in some instances to determine situation-dependent patient-specific data, which allows more precise selection of a value for the cycle duration from a value dataset stored in the storage unit. To determine the cycle duration, provision is made for example for capturing the pulse and/or the blood pressure of the patient using measuring technology, so that it is possible to determine the blood flow and ultimately the circulation period therefrom.

According to one specific embodiment of the control unit the image-generating modality and an evaluation unit serve as a measuring apparatus, with the period duration in particular and therefore the cycle duration of the bloodstream being captured directly. Provision is made here for example for slice images to be generated continuously or at relatively short time intervals in a specified region of the body of the patient with the aid of the image-generating modality, so that a blood vessel for example is monitored over quite a long period in this region. The time interval between the first emergence or first pass of the bolus in the monitored region and the second emergence or second pass is then captured as the cycle duration.

This determination of the cycle duration here in particular does not take place as part of the actual examination but immediately before the actual examination, with a much smaller test dose of the contrast agent being injected during the determination of the cycle duration than the dose for the examination but with the same time pattern for the injection rate as for the actual examination that follows. The corresponding bolus of the test dose here is not suitable for the examination of the organ to be examined but is sufficient to be registered during the determination of the cycle duration in the monitored region.

Alternatively the determination of the cycle duration takes place as part of the actual examination, with it then being determined how much time elapses from the time of the start of the injection of the contrast agent to the emergence of the bolus in a monitored region, for example a blood vessel in proximity to the heart or the region to be examined, and with the cycle duration being determined by multiplying the time interval thus determined by a predefined factor. The distance between the site of the injection and the monitored site in the body of the patient thus serves for this approach as a type of test distance, which is monitored to allow calculation of the cycle duration.

A variant of the control unit is provided, in which the time pattern of the injection rate is embodied in the manner of a pulse sequence, with the time interval between the pulses of the pulse sequence essentially corresponding to the patient-specific cycle duration. The injection of the contrast agent at an injection rate embodied in a pulsed manner has, so the resulting bolus shape, in other words the spatial distribution of the contrast agent concentration in the relevant blood volume, is particularly favorable for image generation using the image-generating modality.

An essentially rectangular shape may be provided for the pulses. Therefore either the value zero or a constant value C is provided for the injection rate. This generates a bolus with a shape in the manner of a bell curve, which moves forward virtually in the manner of a Gaussian package in the blood vessel system of the patient, gradually dispersing in the process. This specific embodiment of the bolus has proven beneficial in particular for examinations, in which the time pattern of the influencing of the image data by the contrast agent in a region to be examined is of interest. One example of this is the examination of the blood supply to an organ, in which it is desirable to distinguish between arteries and veins. The gradient or rather the pattern of the gradient of the bolus here may be predefined by suitable selection of a value for the injection rate. Suitable measuring and evaluation methods, such as “time to peak” or “slope”, are then used during the examination to determine how the pattern of the gradient of the bolus changes as it passes through the blood vessel system.

In an embodiment of the control unit the pulses of a pulse sequence are embodied differently. This means that the region to be examined is flooded with contrast agent in a different manner according to the pulse sequence after every cycle duration so that for example different structures in the region to be examined may be captured using imaging technology during a single examination, different contrast agent concentrations in the tissue being necessary to show them. For example a pulse sequence with at least two rectangular pulses is provided, in which the partial dose of contrast agent administered with each pulse is less than 50% of the partial dose of the previous pulse. In another embodiment, the partial dose of contrast agent administered with each pulse is less than 20% of the partial dose of the previous pulse. The change in the partial doses from pulse to pulse may be achieved here by varying the pulse height and/or by varying the pulse duration. In one embodiment, only the pulse time interval corresponds in each instance to the cycle duration and remains unchanged from pulse to pulse. In another embodiment, a pulse sequence is provided, in which a number of rectangular pulses, each with a time interval corresponding to the cycle duration, have the same pulse width but a pulse height that increases from pulse to pulse. The object here is in particular to reshape the pattern of the gradient of the bolus due to each partial injection of contrast agent in such a manner that it has a predefined basic shape immediately after every partial injection and a predefined rise in particular at least in one segment. This means that virtually the same start values are predefined for every image-generating process of an examination, in which the change in the pattern of the gradient or the change in the rise is determined as a characteristic variable, giving a high level of comparability for the information obtained during every image-generating process.

A variant of the control unit is also expedient, in which the overall dose of contrast agent for the respective data acquisition is predefined. The injection of contrast agent is terminated here in particular independently of the specified time pattern of the injection rate, as soon as the predefined overall dose of contrast agent is reached. If therefore for example a pulse sequence with five identically embodied pulses is provided, and the overall dose of contrast agent is already reached after the third pulse, the injection process is terminated and the remaining pulses are not executed. Since the injection of a contrast agent represents a relevant strain on the body of the patient, compliance with an overall dose of contrast agent is a high priority and the time pattern of the injection rate for the contrast agent is tailored correspondingly to said overall dose of contrast agent. The fact that said overall dose of contrast agent is predefined and monitored by the control unit means that the device arrangement and therefore the medical examination apparatus has a particularly high level of operating safety and inadvertent overdosing is reliably prevented.

According to one development the overall dose of contrast agent is predefined for the respective data acquisition as a function of the patient-specific cycle duration. Until now it has been normal procedure to determine the overall dose of contrast agent for an examination based on patient-specific data, such as for example height and weight. By taking into account the state of the patient, as determined more precisely by the cycle duration, the determination of an overall dose of contrast agent favorable for the patient and the respective data acquisition takes place as a function of the state of the patient and is therefore much more effective.

According to one particularly expedient embodiment of the examination apparatus a central control unit of the image-generating modality is provided as the control unit. The central control unit of the image-generating modality then takes over the additional tasks that are otherwise performed by an independent control unit. Since an image-generating modality frequently comprises a central control unit anyway, this allows the safety concept presented here to be achieved virtually by retrofitting with relatively little outlay, even in the case of medical examination apparatuses that are already in use. In the most favorable instance changes then only have to be made in the area of the data exchange between the individual devices of the examination apparatus and in the control software of the individual components.

Provision is also made, for particularly good compatibility, in one variant of the examination apparatus for standard connections, such as for example CAN bus or Bluetooth, and standard protocols to be used for communication between the control unit, the image-generating modality and the controllable injection apparatus.

### BRIEF DESCRIPTION OF THE DRAWINGS

The embodiments are described in more detail below based on a schematic drawing, in which:

FIG. 1 shows a block diagram of a medical apparatus,

FIG. 2 shows a graphic diagram of a time pattern of an injection rate and a time pattern of a contrast agent concentration and

FIG. 3 shows a graphic diagram of an alternative time pattern of the injection rate and an alternative time pattern of the contrast agent concentration.

### DETAILED DESCRIPTION OF INVENTION

Corresponding parts are shown with identical reference characters in all the figures.

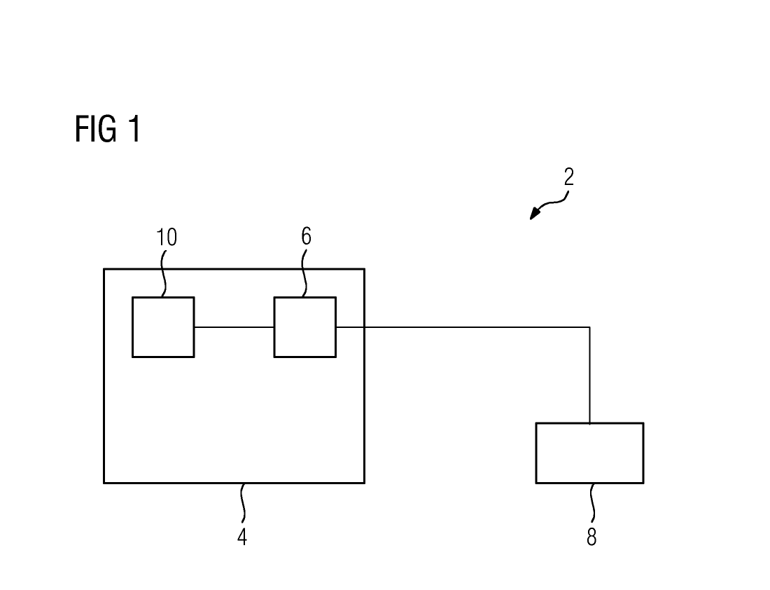

In the case of the medical examination apparatus 2 described below and illustrated schematically in FIG. 1 a magnetic resonance tomography unit 4 with a central control unit 6 is used to examine a patient using an imaging method. Depending on the examination method selected, provision is made for a contrast agent to be injected into the patient to assist the imaging method. For this purpose the medical examination apparatus 2 comprises an injection apparatus 8, may be configured as an injection pump, also referred to as a dosing pump.

The injection apparatus 8 used in the exemplary embodiment is a controllable injection apparatus 8, which is connected for signaling purposes to the control unit 6 of the magnetic resonance tomography unit 4 and which is activated by way of the control unit 6. This tailors the injection of the contrast agent and in particular the time pattern of the injection rate of the contrast agent to the respective examination and in particular to the respective selected image-generation method, in order thereby to influence the quality of the image data to be generated in a positive manner.

Before the start of a corresponding examination the patient is positioned on an examination table of the magnetic resonance tomography unit 4. Also, if as here provision is made for an injection of contrast agent, an injection needle, which is part of the injection apparatus 8, is inserted into the body, typically into a vein, of the patient.

An operator, for example a medical technical assistant (MTA), then uses a console (not shown in detail) to select, for example by inputting a parameter set, a control program for the control unit 6, which determines the order in which the magnetic resonance tomography unit 4 on the one hand and the injection apparatus 8 on the other hand are activated automatically during the examination.

For the examination considered by way of example, provision is made for a two-phase examination with a two-phase injection of contrast agent, with provision being made in each of the two phases for a pulsed injection of a partial dose of contrast agent and an image generation phase starting at a different time therefrom. The two phases here are matched to one another in respect of time, with the time matching taking place on the basis of the monitoring of a region in the body of the patient using the magnetic resonance tomography unit 4. To this end the image data generated during monitoring is analyzed using an evaluation unit 10, in order thus to determine a patient-specific cycle duration, the value of which is taken into account by the central control unit 6 during activation of the injection apparatus 8. The central control unit 6 therefore uses a signal connection to access the analysis data of the evaluation unit 10.

In the exemplary embodiment the organ to be examined is the heart of the patient, with the method presented here and being implemented by means of the central control unit 6 being generally suitable for examining all organs or body regions with a good flow of blood through them. The injection of the contrast agent takes place in the region of a vein in the arm and a cross section of the body of the patient in the region of the aorta is provided as the region to be monitored. When the contrast agent is injected into the vein in the arm, said contrast agent mixes with the blood, with the result that a mixture of contrast agent and blood forms in an initially locally limited manner and moves forward in the direction of the aorta due to the patient's heartbeat, where it is located as a result of monitoring by the magnetic resonance tomography unit 4 by means of the evaluation unit 10 due to the influencing of the image data by the contrast agent. The period between location and the start of the injection is captured using measuring technology and multiplied by a stored factor, in this instance 3. The resulting value corresponds closely to the time taken by a relevant blood volume or the bolus to pass through the entire bloodstream and is therefore taken subsequently into account as the patient-specific cycle time by the central control unit 6 when controlling the magnetic resonance tomography unit 4 and the injection apparatus 8.

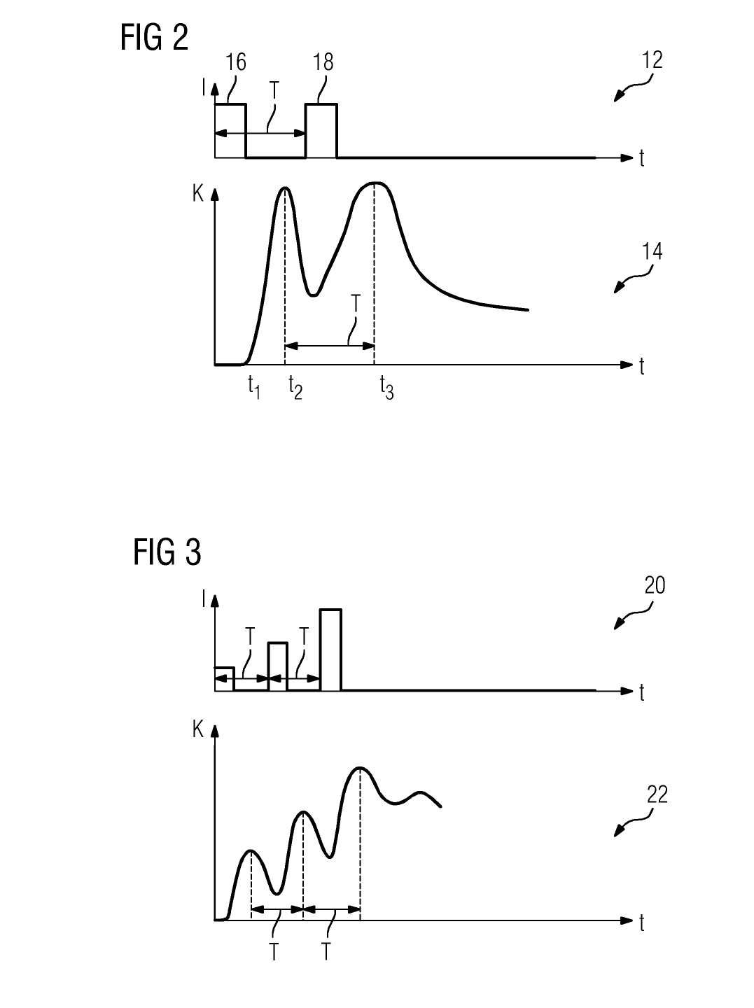

The manner in which the injection apparatus 8 and the magnetic resonance tomography unit 4 are activated is illustrated schematically in FIG. 2. The upper of the two graphs 12 here shows an exemplary change in the injection rate I(t) over time, while the lower graph 14 shows a time pattern of a contrast agent concentration K(t) and therefore virtually the action of the contrast agent. If for example a contrast agent is used, which shortens the T1 relaxation time in the blood or in tissue, in other words for example gadolinium chelate, when the contrast agent arrives in the monitored region the magnetic resonance signal from this very region changes. The change compared with the state without contrast agent allows the concentration of contrast agent in the blood in the monitored region to be determined. The change over time in the concentration of the contrast agent in the blood in the monitored region is outlined in the lower graph 14 in FIG. 2.

It can be seen from the upper graph 12 that the injection of contrast agent is first started with a constant injection rate over the duration of a first pulse 16. After a certain time t₁ the contrast agent injected during the first pulse 16 passes into the monitored region and modifies the measurement signals of the magnetic resonance tomography unit 4 there. The pulsed injection of the contrast agent at an identical injection rate over the entire duration of the pulse means that the contrast agent concentration registered by monitoring initially rises steeply, reaches its maximum at t₂ and then drops relatively steeply again. The locally limited contrast agent/blood mixture then migrates once through the bloodstream of the patient, before returning to the monitored region. Therefore at t₃ a further local maximum contrast agent concentration occurs, which is also referred to as the bolus echo. This local maximum is typically very much smaller due to the dispersal of the bolus compared with the image than the local maximum at t₂. A bolus echo is also characterized by a very much wider curve pattern and a flatter rise.

The time interval between the two maxima, in other words t₃-t₂, corresponds here to the patient-specific cycle time T and therefore to the time taken by a bolus to migrate once through the bloodstream. As already mentioned above, the examination in the present instance is embodied in two phases and the injection of the contrast agent takes place in the form of two injection pulses. The second pulse 18 is started precisely one cycle duration T after the start of the first pulse 16. This means that the contrast agent/blood mixture that moves through the bloodstream and therefore also returns to the position of the injection needle has further contrast agent added when it reaches the position of the injection needle, by the injection of a further partial dose of contrast agent, in other words by the second pulse 18, so that the curve pattern of the bolus echo in the diagram in FIG. 2 is widened but has a comparably large maximum to the curve pattern of the bolus. The bolus is thus as it were refreshed, so it is suitable for the generation of a second examination image.

The generation of an examination image here serves to generate image data, which is used later in an evaluation by a physician to produce a diagnosis and which is therefore the object of said examination. The generation of an examination image is provided for in the region of the heart of the patient in the exemplary embodiment and is to be started when the contrast agent reaches a specified region of the heart, for example the right ventricle. The measurement here should start in particular at the same time as the rise in contrast agent concentration. With the examination described here with the two-phase injection of contrast agent, a correspondingly favorable situation is achieved twice, with the cycle duration T specifically present between the two situations. Two phases are therefore also provided for the generation of the examination image, with an examination image or a series of examination images being generated in each. Each image-generating examination is started by the control unit 6, which accesses the evaluation data of the evaluation unit 10 for this purpose. In this process the registration of a maximum contrast agent concentration at t₂ brings about the release of a trigger function, which starts the generation of the examination image after a predefined delay time.

As an alternative to the two-phase embodiment of the patient examination, multi-phase examinations are also provided, a three-phase variant being described in more detail below. With this variant a sequence of three pulses is provided for the time pattern of the injection rate I(t) of the contrast agent, as shown in the upper graph 20 in FIG. 3. As in the previous example, the time interval between two successive pulses of said pulse sequence is defined by the cycle duration T. However the pulses of this pulse sequence have different pulse heights, in contrast to the previous example, with the pulse height doubling here from pulse to pulse by way of example. A partial dose of contrast agent is injected into the body of the patient with every pulse, being defined by the pulse height on the one hand and the pulse duration on the other hand. The sum of all the partial doses of contrast agent is the overall dose of contrast agent, the value of which is determined prior to the examination and stored in a storage unit, to which the control unit 6 has access. As soon as the control unit 6 determines that the overall dose of contrast agent has been reached, it stops the injection apparatus 8, regardless of whether or not the planned injection, in this instance the pulse sequence, has been completed.

The lower graph 22 in FIG. 3 again shows the time pattern of contrast agent concentration K(t) in the monitored region. The increasing pulse height in the pulse sequence means that the value of the local maximum after each cycle duration T also increases. It is essential here however that the pattern of contrast agent concentration K(t) has an approximately identical gradient pattern after every injection of a partial dose of contrast agent.

The invention is not restricted to the exemplary embodiment described above. Instead the person skilled in the art can derive different variants therefrom, without departing from the disclosed subject matter. In particular all the individual features described in relation to one or more embodiment can also be combined differently with one another, without departing from the subject matter of the invention.

1. A control unit for a device arrangement, comprising:
an image-generating modality; and
a controllable injection apparatus for a contrast agent,
wherein an injection rate of the injection apparatus is varied during data acquisition as a function of a patient-specific cycle duration, which in particular indicates a time period during which a blood volume is conveyed once through the bloodstream of a patient.

2. The control unit as claimed in claim 1,
wherein the patient-specific cycle duration is predefined for the respective data acquisition.

3. The control unit as claimed in claim 1,
wherein a measuring apparatus is provided to determine the patient-specific cycle duration for the respective data acquisition.

4. The control unit as claimed in claim 3,
wherein the image-generating modality and an evaluation unit serve as the measuring apparatus.

5. The control unit as claimed in claim 1,
wherein the time pattern of the injection rate is embodied in the manner of a pulse sequence, and
wherein the time interval between the pulses of the pulse sequence corresponds essentially to the patient-specific cycle duration.

6. The control unit as claimed in claim 5,
wherein an essentially rectangular shape is provided for the pulses.

7. The control unit as claimed in claim 5,
wherein the pulses of a pulse sequence are embodied differently.

8. The control unit as claimed in claim 7,
wherein the partial dose of contrast agent defined by the respective pulse increases from pulse to pulse in a pulse sequence.

9. The control unit as claimed in claim 1,
wherein the overall dose of contrast agent is predefined for the respective data acquisition.

10. The control unit as claimed in one of claim 1,
wherein the overall dose of contrast agent is predefined for the respective data acquisition as a function of the patient-specific cycle duration.

11. The control unit as claimed in claim 1,
wherein standard protocols are used for communication with the device arrangement.

12. The control unit as claimed in claim 1, comprises:
a central control unit of the image-generating modality.

13. A medical examination apparatus comprising a control unit as claimed in claim 1.
